(19) United States
(12) Patent Application Publication
(10) Pub. No.: US 2014/0200341 A1
(43) Pub. Date: Jul. 17, 2014

(54) Method for the Production of Fluoromethyl Esters of Androstan-17 beta Carboxylic Acids

(71) Applicant: HOVIONE INTER. LTD, Lucerne 7 (CH)

(72) Inventors: Emilia Perpetua Tavares Leitao, Sao Marcos (PT); Maria Rita Ventura, Lisboa (PT); Christopher Maycock, Carcavelos (PT)

(73) Assignee: HOVIONE INTER LIMITED, Lucerne 7 (CH)

(21) Appl. No.: 14/122,058

(22) PCT Filed: May 25, 2012
(86) PCT No.: PCT/GB2012/000469

§ 371 Date: Feb. 14, 2014

(30) Foreign Application Priority Data
May 26, 2011 (PT) 105723

(51) Int. Cl. C07J 17/00; C07J 31/00

(52) U.S. Cl. CPC C07J 17/00; C07J 31/006
USPC 540/114; 552/610

(57) ABSTRACT

Described herein are processes for the preparation of monofluoromethylated organic biologically active compounds, starting from protected intermediates and/or reagents to obtain compounds such as fluticasone propionate and fluticasone furoate, in presence of decarboxylating reagents XeF₂ and BrF₃, or using FCH₂SH as a reagent.

The present invention describes processes for the preparation of monofluoromethylated organic biologically active compounds, starting from protected intermediates and/or reagents to obtain compounds such as Fluticasone Propionate and Fluticasone Furoate, in presence of decarboxylating reagents XeF₂ and BrF₃, or using FCH₂SH as a reagent.

### BACKGROUND TO THE INVENTION

The carbon-fluorine bond is commonly found in pharmaceutical and agrochemical products, because it is generally metabolically stable and the fluorine atom acts as a bioisostere of the hydrogen atom (Ann M. Thayer “Fabulous Fluorine” Chemical and Engineering News, Jun. 5, 2006, Volume 84, pp. 15-24). Nowadays around 200 of all pharmaceutical compounds and 30-40% of agrochemicals on the market contain fluorine. Fluorination and fluoroalkylation are the two major synthetic methods to prepare selectively fluorinated organic compounds. Monofluoromethylation (selective introduction of a CH₂F group into the organic molecule) is less studied than fluorination. The exploration of di- and monofluoromethylated compounds as organic biologically active compounds has emerged recently. As a result, a variety of structurally diverse —CH₂F containing drugs have been developed, such as: Afloqualone, Fluticasone Propionate (Jinbo Hu, Wei Zhang, and Fei wang, Chem. Commun., 2009, 7465-7478), Fluticasone Furoate and the anaesthetic Sevoflurane. The efficient and selective incorporation of monofluoromethylated moieties into the organic molecule is beneficial for the synthesis of the target molecule. The process is usually carried out directly using CH₂FBr or indirectly, using CH₂BrI or CH₂ClI, among others. These compounds are known as hydrohalofluorocarbons or freons (HCFCs), which is a subclass of chlorofluorocarbons (CFCs). Every permutation of fluorine, chlorine, and hydrogen on the methane and ethane template has been examined and most have been commercialized. Furthermore, many examples containing bromine are known for higher numbers of carbon as well as related compounds. The use of this class of compounds include refrigerants, blowing agents, propellants in medicinal applications, and degreasing solvents (M. Rossberg et al. “Chlorinated Hydrocarbons” in Ullmann's Encyclopedia of Industrial Chemistry, 2006, Wiley-VCH, Weinheim).

Unfortunately, due to their high stability, CFCs do not decompose in the lower atmosphere as many industrial chemicals do. In fact they are accumulating and eventually rise to the stratosphere. Ultraviolet radiation in the stratosphere breaks the CFCs apart, and the released chlorine atoms destroy the ozone in upper atmosphere. For this reason, the manufacture of such compounds is being phased out according to the Montreal Protocol (Pool, R. 1989. The elusive replacements for CFCs. Science 242: 666). Under the Montreal Protocol, it was agreed to start reducing its consumption and production in 2015.

The literature describes a method for replacing a carboxylic group with a fluorine group in a halogenated aliphatic carboxylic compound having the general formula, R—COOH, to prepare a fluorinated product having the general formula, R—F. The fluorodecarboxylation is carried out in the presence of XeF₂ (Timothy B. Patrick, Kamalesh K. Johri, David H. White, William S. Bertrand, Rodziah Mokhtar, Michael R. Kilbourn, and Michael J. Welch, Can. J. Chem., Vol. 64, 1986, 138) or BrF₃ (U.S. Pat. No. 4,996,371).

Copending patent application PT105138 describes the application of these reagents, for example, in the synthesis of Fluticasone Propionate and Fluticasone Furoate, as depicted in Scheme 1 below, hence avoiding the use of bromofluoromethane or any other related substance that deplete the ozone layer.

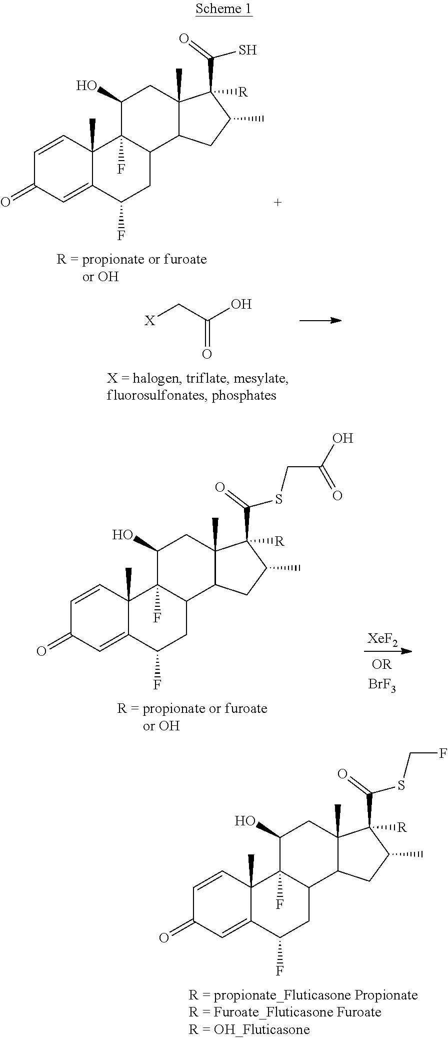

### DETAILED DESCRIPTION OF THE INVENTION

However, we now have surprisingly found that better results are obtained, contrary to what is described in the prior art, when the hydroxyl group at the C-11 position, in the steroid is protected, and/or when the acetic acid group is protected as an ester or when combinations of protected and non-protected compounds are used, for example, as depicted in Schemes 2 and 3 below.

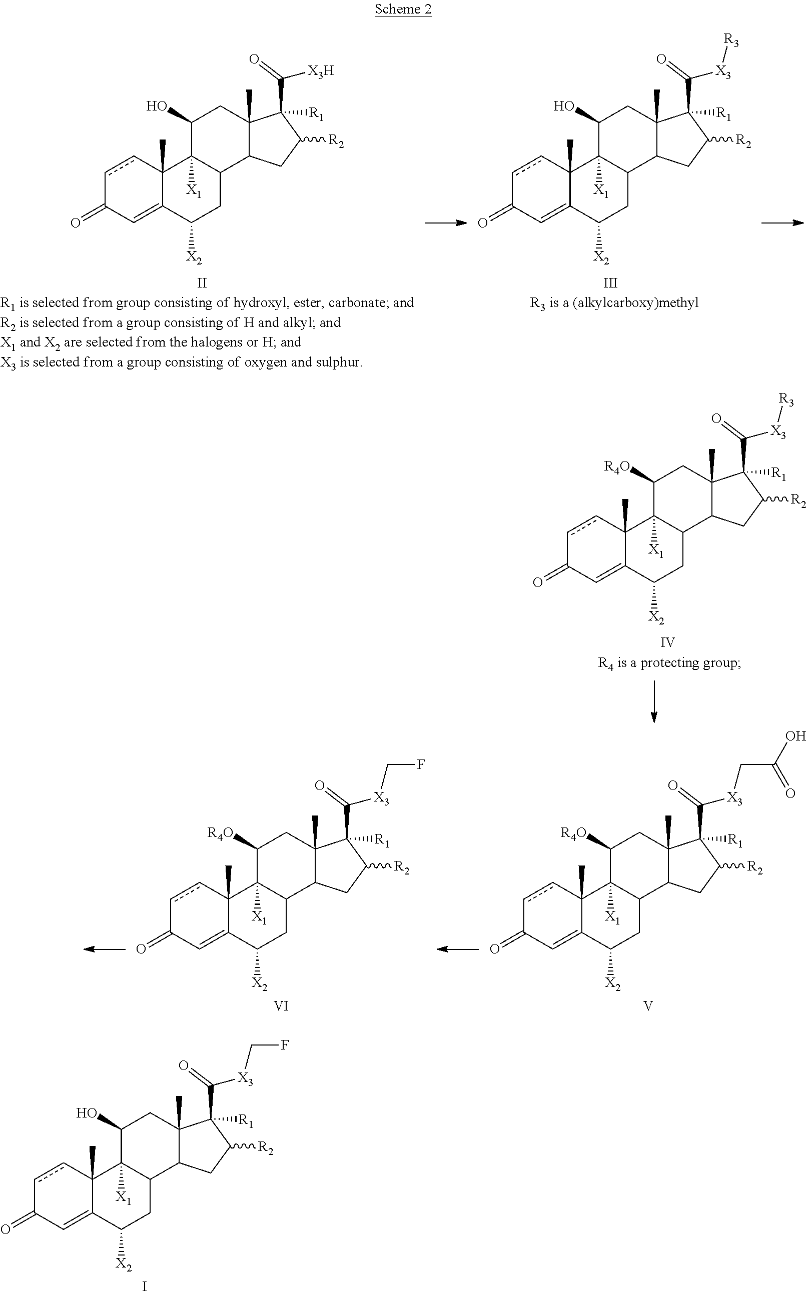

Scheme 2 illustrates the reaction of steroid (II), with a carboxymethyl ester to afford intermediate (III). The hydroxyl group in the C-11 position is protected to yield a Intermediate (IV), which is hydrolyzed to obtain the corresponding free carboxylic acid (V), which is then fluorodecarboxylated to obtain compound of formula (VI) and deprotected to obtain compound of formula (I). The order and number of steps is not limited to the scheme presented above.

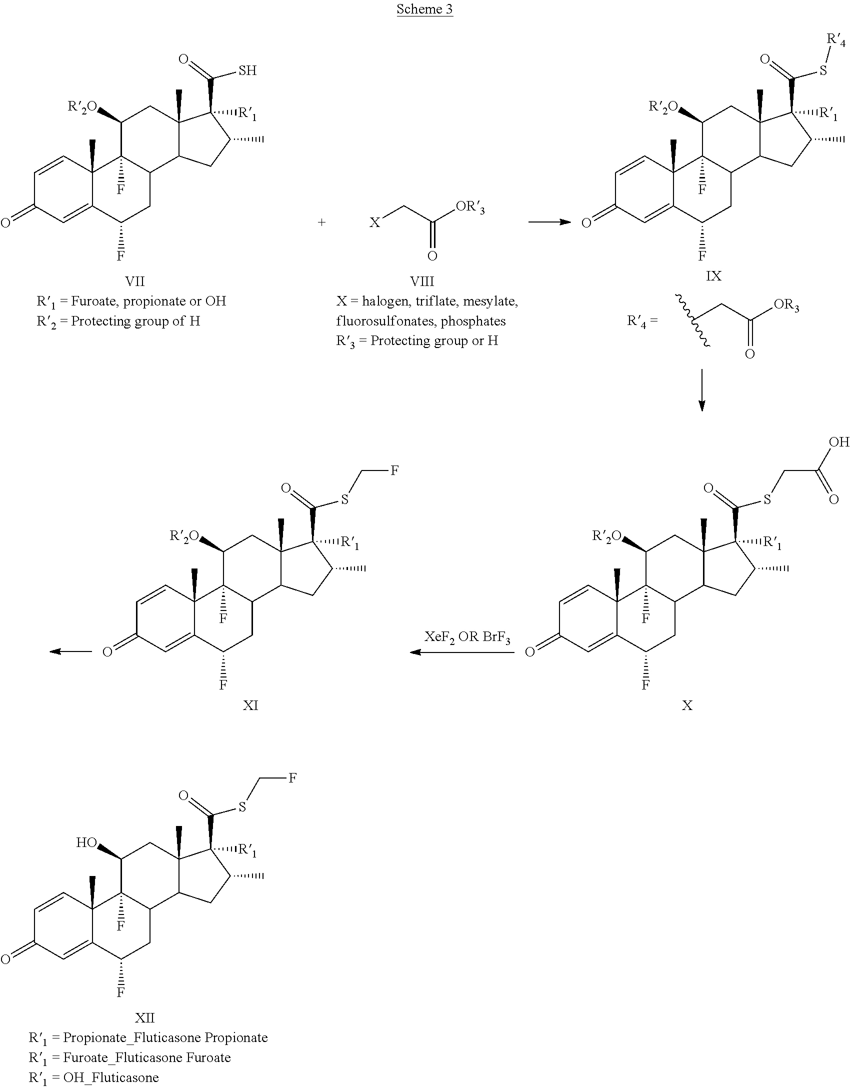

Scheme 3 illustrates the reaction of steroid (VII), with X-acetic acid or X-acetic ester (VIII) to afford intermediate (IX). Intermediate (IX) is converted to the free carboxylic acid (X) which is then fluorodecarboxylated to obtain compound of formula (XI) and deprotected to obtain compound of formula (XII): Fluticasone, Fluticasone Propionate or Fluticasone Furoate. The order and number of steps is not limited to the scheme presented above.

Thus, according to a first aspect of the present invention, there is provided a method of preparing a biologically active organic compound of formula (I),

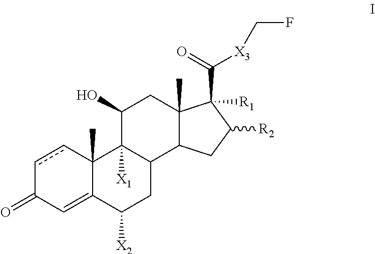

wherein:
R₁ is selected from group consisting of hydroxyl, ester and carbonate; and
R₂ is selected from a group consisting of H and alkyl; and
X₁ and X₂ are selected from the group consisting of H and halogen; and
X₃ is selected from a group consisting oxygen and sulphur;
which method comprises one or more of the following steps:
(a) reacting a compound of formula (II) with an ester of formula X—CH₂C(O)OR′₃ to yield a compound of formula (III),

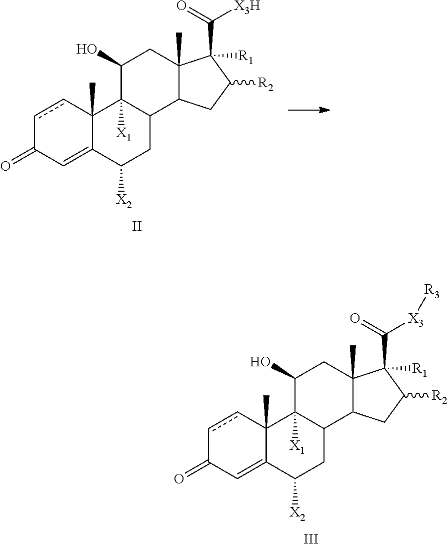

wherein:
R′₃ is an alkyl group;
R₃ is an (alkylcarboxy)methyl group;
X is a leaving group selected from halogen, triflate, mesylate, fluorosulfonate and phosphonate; and
R₁, R₂, X₁, X₂ and X₃ are as defined with reference to formula (I); and/or
(b) protecting the C-11 position of a compound of formula (III) to yield a compound of formula (IV),

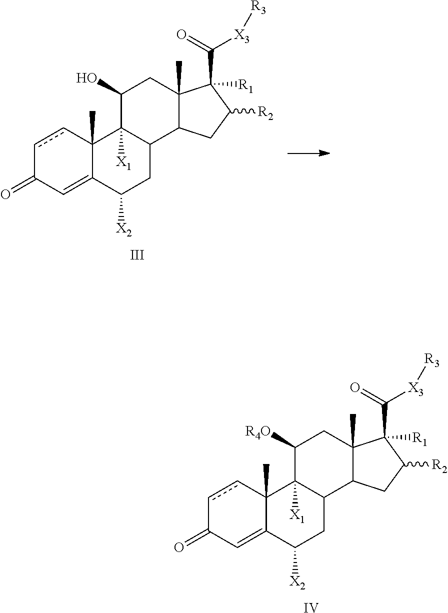

wherein:
R₄ is a suitable protecting group; and
R₁, R₂, R₃, X₁, X₂ and X₃ are as defined with reference to formulae (I) and (III); and/or
(c) deprotecting the ester at the C-21 position of a compound of formula (IV) to yield a compound of formula (V),

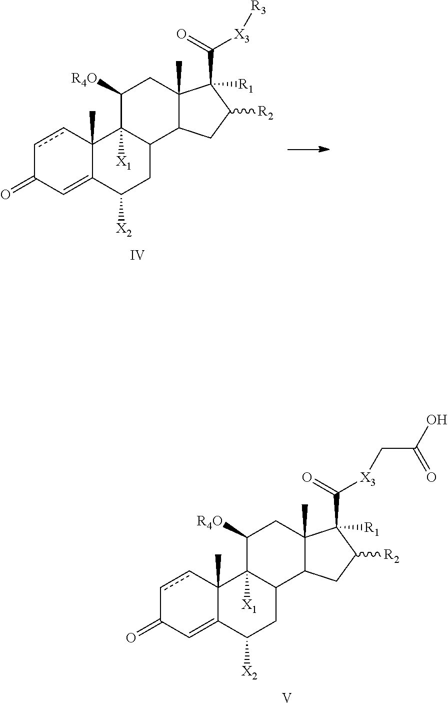

wherein:
R₁, R₂, R₃, R₄, X₁, X₂ and X₃ are as defined with reference to formulae (I), (III) and (IV); and/or
(d) reacting a compound of formula (V) with a suitable fluorodecarboxylating agent to yield a compound of formula (VI),

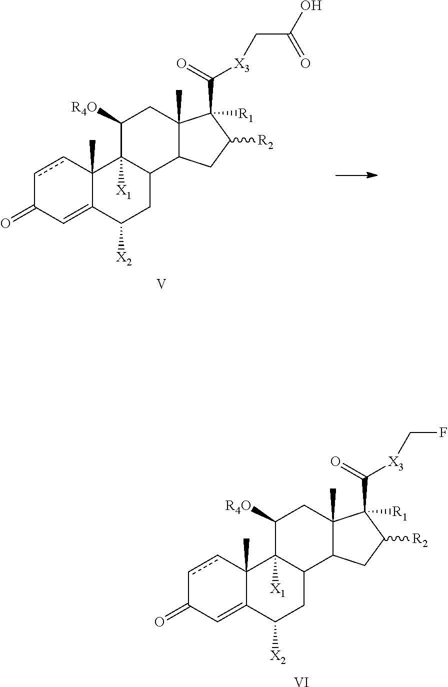

wherein:
R₁, R₂, R₄, X₁, X₂ and X₃ are as defined with reference to formulae (I), (III) and (IV); and/or
(e) hydrolysing the C-11 protecting group of a compound of formula (VI) to yield a compound of formula (I).

In one embodiment of the invention, R₁ represents hydroxyl or an ester group of formula —OC(O)R′, wherein R′ represents an alkyl of aryl group. Preferably, R′ represents a linear or branched chain alkyl group, more preferably a linear or branched chain C₂₋₆ alkyl group, and most preferably a linear or branched chain C₁₋₄ alkyl group, such as methyl, ethyl n-propyl, iso-propyl, n-butyl, sec-butyl, iso-butyl and tert-butyl, preferably ethyl. When R′ represents an aryl group, it is preferably a C₃₋₆ aryl group, optionally containing one or more heteroatoms, such as phenyl, furan or thiophene. In a particularly preferred embodiment, R′ represents ethyl or thiophene; i.e. R₁ represents propionate or furoate.

When R₂ represents an alkyl group, it is preferably a linear or branched chain C₁₋₈ alkyl group, more preferably a linear or branched chain C₁₋₆ alkyl group, and most preferably a linear or branched chain C₁₋₄ alkyl group. Preferred examples of R₂ include methyl, ethyl, n-propyl, iso-propyl, n-butyl, sec-butyl, iso-butyl and tert-butyl. More preferred examples of R₂ include methyl and ethyl, especially methyl. In an alternative preferred embodiment, R₂ is H.

The alkyl group of the (alkylcarboxy)methyl substituent R₃ is preferably a linear or branched chain C₁₋₈ alkyl group, more preferably a linear or branched chain C₁₋₆ alkyl group, and most preferably a linear or branched chain C₁₋₄ alkyl group. Preferred examples include methyl, ethyl, n-propyl, iso-propyl, n-butyl, sec-butyl, iso-butyl and tert-butyl. A particularly preferred example is tert-butyl.

As used herein the term “organic biologically active compound” means an organic compound which is of medicinal or therapeutic use in the broadest sense. Typically, the organic biologically active compound is a pharmaceutically active compound.

As used herein the term “halogen” means F, Cl, Br or I. In a preferred embodiment of the invention, both X₁ and X₂ represent F.

In one embodiment of the invention, X₃ represents O. In an alternative embodiment of the invention, X₃ represents S.

Particularly preferred examples of a compound of formula (I) include, but are not limited to, Fluticasone, Fluticasone Propionate and Fluticasone Furoate.

In one embodiment of the invention, there is provided a method of preparing a biologically active organic compound of formula (I), which method comprises performing reaction step (a) as hereinbefore defined and optionally one or more of steps (b), (c), (d) and (e).

In an alternative embodiment of the invention, there is provided a method of preparing a biologically active organic compound of formula (I), which method comprises performing reaction step (b) as hereinbefore defined and optionally one or more of steps (a), (c), (d) and (e).

In alternative embodiment of the invention, there is provided a method of preparing a biologically active organic compound of formula (I), which method comprises performing reaction step (c) as hereinbefore defined and optionally one or more of steps (a), (b), (d) and (e).

In alternative embodiment of the invention, there is provided a method of preparing a biologically active organic compound of formula (I)_, which method comprises performing reaction step (d) as hereinbefore defined and optionally one or more of steps (a), (b), (c) and (e).

In alternative embodiment of the invention, there is provided a method of preparing a biologically active organic compound of formula (I), which method comprises performing reaction step (e) as hereinbefore defined and optionally one or more steps (a), (b), (c) and (d).

Suitable protecting groups for use in the present invention are commercially available and/or may be prepared by methods known in the art. Preferred examples include, but are not limited to, trifluoroacetate, acetate and trichloroacetate.

Suitable deprotection reagents for use in the present invention are commercially available and/or may be prepared by methods known in the art. Preferred examples include, but are not limited to, trifluoroacetic anhydride (TFA), triethylamine, pyridine and Hunig's base.

Suitable fluorodecarboxylating agents for use in the present invention are commercially available and/or may be prepared by methods known in the art. Preferred examples include, but are not limited to, XeF₂ and BrF₃.

Intermediate (III) can be prepared by the reaction of steroid (II) with a carboxylmethyl ester in an organic solvent and in presence of organic or inorganic base at a temperatures range within −70° C. and 70° C. The product can be isolated by precipitation in water or water with acid or water with base, by extraction with organic solvent and/or concentration, by recrystallization in organic solvent, and/or by column chromatography. Resin and activated charcoal can also be used during the work-up to purify the product.

The intermediate (IV) can be prepared in the same conditions as intermediate (III), protecting the C-11 position with a protecting group. Intermediate (V) can be prepared in the same conditions as intermediate (III), deprotecting the ester in C-21 position of formula (IV) to yield a compound of formula (V). Intermediate (VI) can be prepared in the same conditions as intermediate (III), in the presence of XeF₂ or BrF₃ by fluorodecarboxylation. Intermediate (VI) is then hydrolyzed to give compound of formula (I).

The use of substances that deplete the ozone layer, such as: CH₂BrF, CH₂ClF, CH₂FI, and the like can also be avoided if FCH₂SH or any of its intermediates are used. These reagents will allow the introduction of CH₂F group into the respective organic molecules to obtain of Fluticasone, Fluticasone Propionate or Fluticasone Furoate.

Scheme 4 illustrates a preferred example of a three step reaction according to the present invention, where fluoromethanethiol is prepared in situ, starting from 2-mercaptoacetic acid, and reacts then with an intermediate to afford Fluticasone, Fluticasone Propionate or Fluticasone Furoate.

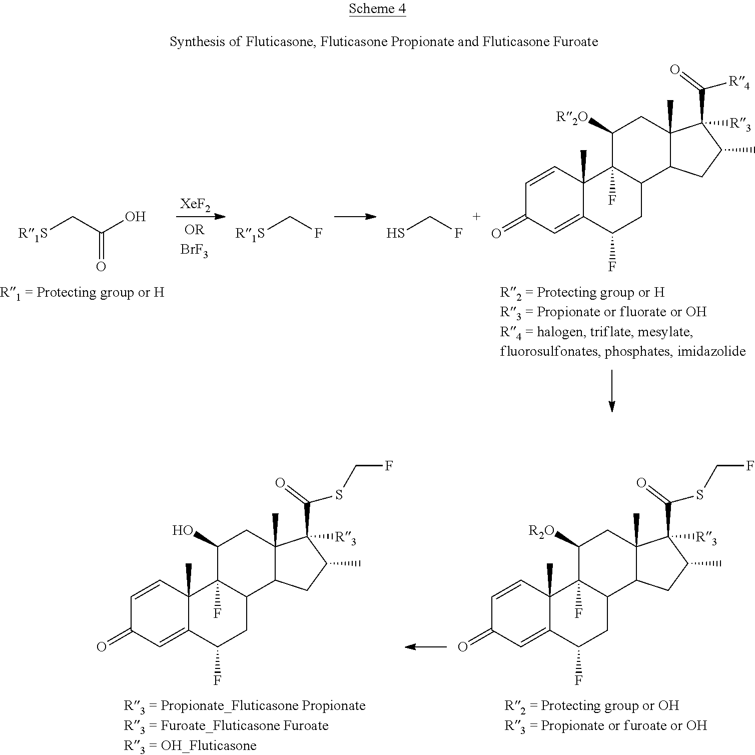

The reaction is not limited to the number of steps presented above.

The intermediate compounds of formulae (III), (IV), (V) and (VI) as described herein are understood to be novel and therefore form a further aspect of the present invention.

According to still a further aspect of the present invention, there is provided the use of a compound of formulae (III), (IV), (V) and/or (VI) for the preparation of a biologically active organic compound containing a monofluoromethylated “—CH₂F” moiety, in particular a compound of formula (I) as described herein, preferably Fluticasone, Fluticasone Propionate and/or Fluticasone Furoate.

### EXAMPLES

The following equipment was employed to analyse the examples of the invention:
NMR—Bruker Avance II 400 MHz: spectra were recorded in CDCl₃.
Infra-red spectra—Mattson Research Series FTIR: spectra were acquired using KBr pellets.
Melting points—Buchi Melting Point B-540.

### Example 1

### Preparation of Fluticasone Propionate

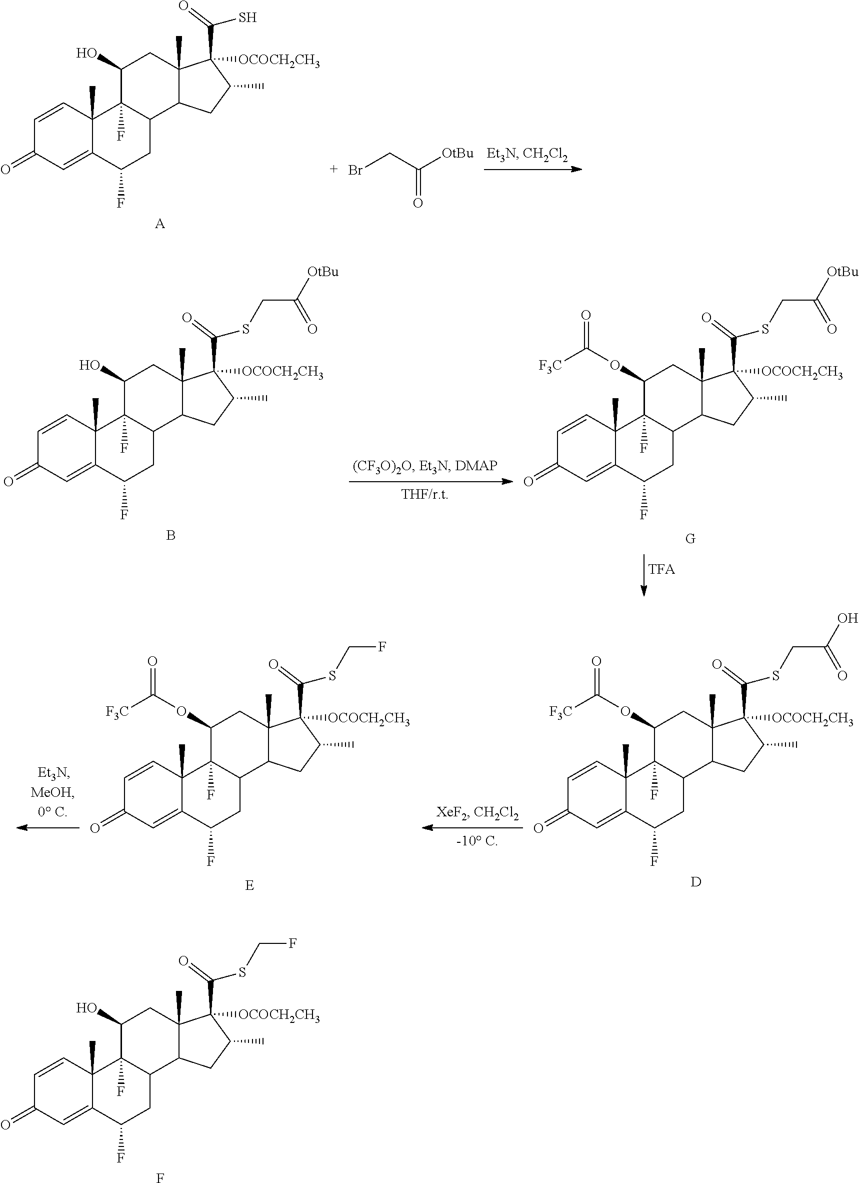

A solution of thioacid steroid, compound of formula A, (5 g, 9.5 mmol), triethylamine (2.2 mL, 14.2 mmol), tert-butyl bromoacetate (1.55 mL, 10.45 mmol) in dichloromethane (15 mL) was stirred at room temperature for 3 h. Water was added (10 mL) and the mixture extracted with dichloromethane (3×20 mL), dried with anhydrous MgSO₄, and concentrated to afford compound of formula B (5.6 g) as a solid.

A solution of compound of formula B (5.53 g, 9.5 mmol), triethylamine (3.29 mL, 23.7 mmol), trifluoroacetic anhydride (TFA) (3.29 mL, 23.7 mmol) and a catalytic amount of DMAP in THF (20 mL) was stirred at room temperature overnight. Water was added (15 mL) and the mixture extracted with ethyl acetate (3×20 mL), dried with anhydrous MgSO₄, and concentrated. Purification by flash column chromatography (1:9 AcOEt/hexane-4:6 AcOEt/hexane) afforded compound of formula C (6.4 g) as a solid.

A solution of compound of formula C (6.4 g, 9.4 mmol) in TFA (15 mL) was stirred at room temperature for 2 h. Evaporation of TFA afforded compound of formula D as a solid.

To a compound of formula D (0.5 g, 0.80 mmol) in dichloromethane (40 mL) at −10° C. it was added XeF₂ (0.260 g, 1.6 mmol) and the solution was stirred at −10° C. for 2 days. 5% NaHCO₃ aqueous solution was added (40 mL) and the mixture extracted with dichloromethane (3×30 mL), dried with anhydrous MgSO₄, and concentrated to afford a crude mixture containing compound of formula E (0.390 g) as a solid foam which was not purified. This crude mixture in MeOH (2 mL) was treated with 1M NH₃ solution in MeOH (0.783 mL) at 0° C. After 5 min, the volatiles were evaporated and flash column chromatography of the residue (1:9 AcOEt/hexane-5:5 AcOEt/hexane) afforded compound of formula F (0.041 g, 10%, 2 steps) as a white solid. ¹H-NMR (CDCl₃), 400 MHz: δ 7.11 (1H, d, J=10 Hz), 6.44 (1H, s), 6.38 (1H, d, J=10 Hz), 5.93 (1H, dd, J=33.6 Hz, J=9.4 Hz), 5.80 (1H, dd, J=33.6 Hz, J=9.3 Hz), 5.38 (1H, ddd, J=49.4, J=11.4, J=6.4 Hz), 4.43-4.41 (1H, m), 3.41-3.38 (1H, m), 2.40-2.26 (6H, m), 1.92-1.73 (4H, m), 1.52 (3H, s), 1.37-1.31 (1H, m), 1.13 (3H, t, J=7.5 Hz), 1.09 (3H, s), 0.99 (3H, d, J=7.2 Hz). ¹³C NMR (CDCl₃), 100 MHz: δ 193.0, 185.5, 172.9, 161.2, 161.1, 150.3, 130.3, 121.2, 121.1, 99.5, 97.8, 96.2, 86.4 (J_{CF}=183 Hz), 80.8 (J_{CF}=215 Hz), 72.0, 71.6, 48.5, 48.0, 47.8, 43.0, 36.5, 36.2, 34.0, 33.7, 33.5, 32.8, 32.7, 32.6, 32.5, 27.5, 23.0, 23.0, 17.1, 16.4, 9.0.

### Example 2

### Preparation of Fluticasone Furoate

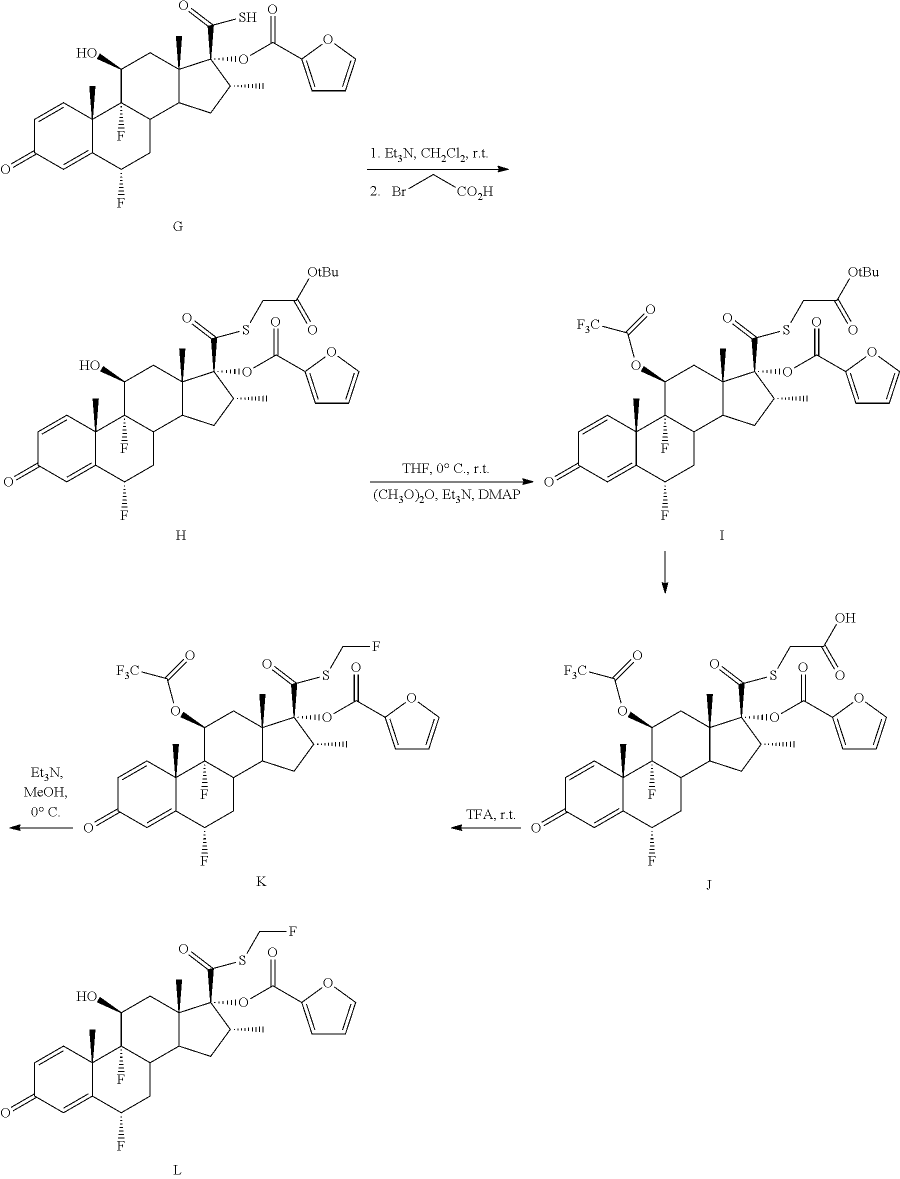

A solution of thioacid steroid, compound of formula G, (5 g, 9.87 mmol), triethylamine (2.05 mL, 14.8 mmol), tert-butyl bromoacetate (1.6 mL, 10.8 mmol) in dichloromethane (20 mL) was stirred at room temperature for 3 h. Water was added (15 mL) and the mixture extracted with dichloromethane (3×20 mL), dried with anhydrous MgSO₄, and concentrated to afford compound of formula H (6.1 g) as a solid foam. Mp=229° C. ¹H NMR (CDCl₃), 400 MHz: δ 7.58 (1H, s), 7.18 (1H, d, J=10.1 Hz), 7.12 (1H, d, J=3.4 Hz), 6.50-6.49 (1H, m), 6.45 (1H, s), 6.40 (1H, d, J=10.1 Hz), 5.40 (1H, ddd, J=48.9 Hz, J=11.4, J=6.4 Hz), 4.45 (1H, d, J=7.5 Hz), 3.72 (1H, d, J=16.1 Hz), 3.62 (1H, d, J=16.1 Hz), 3.47-3.44 (1H, m), 2.51-2.28 (4H, m), 2.16 (1H, broad s), 2.02-1.80 (3H, m), 1.55 (3H, s), 1.47 (9H, s), 1.32-1.35 (1H, m), 1.17 (3H, s), 1.06 (3H, d, J=7.1 Hz). ¹³C NMR (CDCl₃), 100 MHz: δ 194.9, 185.5, 167.8, 161.3, 161.2, 156.9, 150.6, 147.1, 143.7, 130.2, 121.2, 121.1, 118.8, 112.0, 99.8, 98.0, 96.9, 86.5 (J_{CF}=183 Hz), 82.4, 71.9, 71.5, 49.0, 46.0, 43.1, 36.6, 36.4, 33.9, 33.8, 33.6, 33.0, 32.9, 32.8, 32.7, 32.6, 27.9, 23.1, 23.0, 17.2, 16.2. FT-IR (KBr): 3355, 1741, 1725, 1685, 1666, 1621, 1608 cm⁻¹.

A solution of compound of formula H (6.1 g, 9.8 mmol), triethylamine (3.40 mL, 24.5 mmol), trifluoroacetic anhydride (3.44 mL, 24.5 mmol) and a catalytic amount of DMAP in THF (20 mL) was stirred at room temperature overnight. Water was added (15 mL) and the mixture was extracted with CH₂Cl₂ (3×20 mL), dried with anhydrous MgSO₄, and concentrated to afford compound of formula I (7.0 g) as a solid foam. Mp=77-78° C. ¹H NMR (CDCl₃), 400 MHz: δ 7.59 (1H, s), 7.13 (1H, d, J=3.4 Hz), 6.72 (1H, d, J=10.1 Hz), 6.52-6.50 (2H, m), 6.45 (1H, d, J=10.2 Hz), 5.62-5.61 (1H, m), 5.38 (1H, ddd, J=48.5 Hz, J=11.2, J=6.1 Hz), 3.66 (2H, s), 3.49-3.44 (1H, m), 2.62-2.34 (4H, m), 2.21 (1H, d, J=15.4 Hz), 1.96-1.80 (3H, m), 1.43 (9H, s), 1.39 (3H, s), 1.39-1.34 (1H, m), 1.09 (3H, d, J=7.1 Hz), 1.06 (3H, s). ¹³C NMR (CDCl₃), 100 MHz: δ 194.7, 184.9, 167.5, 159.9, 159.7, 156.6, 155.6, 155.2, 148.0, 147.2, 143.5, 131.3, 121.6, 121.5, 119.0, 112.1, 98.2, 96.4, 95.5, 85.9 (J_{CF}=485 Hz), 82.4, 75.6, 75.2, 67.9, 54.5, 48.5, 47.2, 46.5, 46.1, 42.5, 36.6, 33.7, 33.5, 33.3, 33.2, 33.1, 33.0, 32.9, 32.5, 27.7, 25.5, 22.4, 22.3, 17.1, 15.7. FT-IR (KBr): 1789, 1735, 1673, 1639, 1600, 1579 cm⁻¹.

A solution of compound of formula I (7.0 g, 9.77 mmol) in TFA (15 mL) was stirred at room temperature for 2 h. Evaporation of TFA afforded compound of formula J as a white solid (6.5 g). ¹H NMR (CDCl₃), 400 MHz: δ 7.61 (1H, s), 7.16 (1H, d, J=3.4 Hz), 6.89 (1H, d, J=10.0 Hz), 6.59-6.53 (3H, m), 5.63-5.62 (1H, m), 5.41 (1H, ddd, J=48.3 Hz, J=11.2, J=6.3 Hz), 3.85 (1H, d, J=16.6 Hz), 3.79 (1H, d, J=16.6 Hz), 3.50-3.44 (1H, m), 2.61-2.38 (4H, m), 2.18 (1H, d, J=15.2 Hz), 1.99-1.81 (2H, m), 1.41 (3H, s), 1.36-1.30 (1H, m), 1.09 (3H, d, J=7.0 Hz), 1.05 (3H, s). ¹³C NMR (CDCl₃), 100 MHz: δ 194.6, 186.8, 173.8, 159.9, 159.5, 159.1, 158.7, 157.1, 156.1, 155.6, 155.2, 150.6, 147.6, 143.1, 130.5, 121.0, 120.8, 119.5, 112.2, 98.4, 96.7, 96.3, 85.9 (J_{CF}=185 Hz), 75.5, 75.0, 67.9, 54.6, 52.1, 48.4, 47.7, 47.5, 46.7, 46.6, 44.1, 42.5, 36.7, 33.6, 33.5, 33.3, 33.2, 33.1, 33.0, 32.9, 32.5, 31.4, 22.0, 21.9, 16.9, 15.7. FT-IR (KBr): 3498, 1737, 1673, 1637, 1610, 1577 cm⁻¹.

To a compound of formula J (0.050 g, 0.075 mmol) in dichloromethane (4 mL) at −20° C. it was added xenon difluoride (0.024 g, 0.15 mmol) and the solution was stirred at −20° C. for 2 days. 5% NaHCO₃ aqueous solution was added (5 mL) and the mixture extracted with dichloromethane (3×4 mL), dried with anhydrous (MgSO₄) and concentrated to afford a mixture containing compound of formula K (0.044 g) as a white foam. This crude mixture was dissolved in MeOH (1 mL) and it was treated with 1M NH₃ solution in MeOH (0.090 mL) at 0° C. After 5 min, the volatiles were evaporated and the crude mixture was purified by column chromatography yielding the compound of formula L.

It is evident to one skilled in the art that this invention is not limited to the forgoing examples, and that can be embodied in other specific forms without departing from the scope of the invention. Thus, the examples should be considered as illustrative and not restrictive, reference being made to the claims, and that all changes which come within the meaning and range of equivalency of claims be embraced therein.

1. A method of preparing an organic biologically active compound of formula (I), 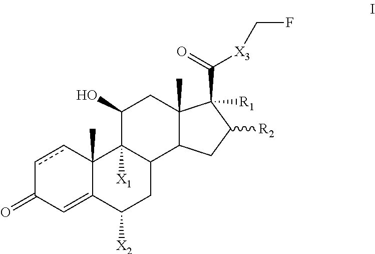
wherein:
R₁ is selected from group consisting of hydroxyl, ester and carbonate;
R₂ is selected from a group consisting of H and alkyl;
X₁ and X₂ are selected from the group consisting of H and halogen; and
X₃ is selected from a group consisting oxygen and sulphur;
which method comprises the step of protecting the C-11 position of a compound of formula (III) to yield a compound of formula (IV), 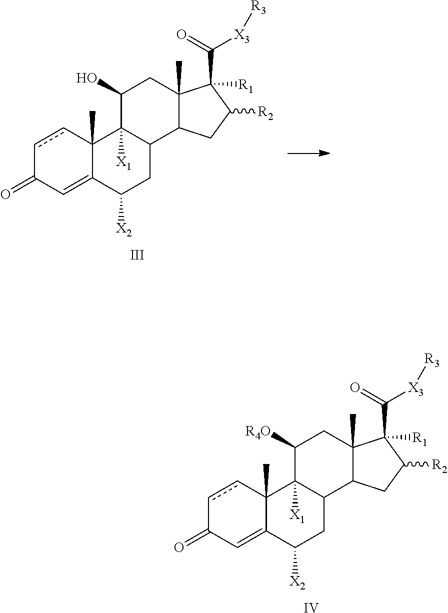
wherein:
R₄ is a suitable protecting group; and
R₁, R₂, R₃, X₁, X₂ and X₃ are as defined with reference to formulae (I) and (III);
and optionally, one or more of the following steps:
(a) reacting a compound of formula (II) with an ester of formula X═CH₂C(O)OR′₃ to yield a compound of formula (III), 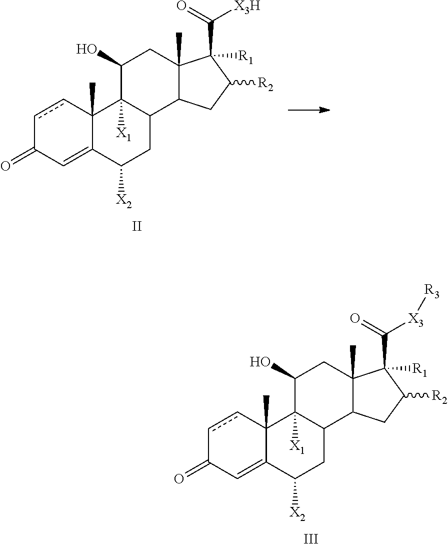
wherein:
R′₃ is an alkyl group;
R₃ is a (alkylcarboxy)methyl group;
X is a leaving group selected from halogen, triflate, mesylate, fluorosulfonate and phosphonate; and
R₁, R₂, X₁, X₂ and X₃ are as defined with reference to formula (I); and/or
(b) deprotecting the ester at the C-21 position of a compound of formula (IV) to yield a compound of formula (V), 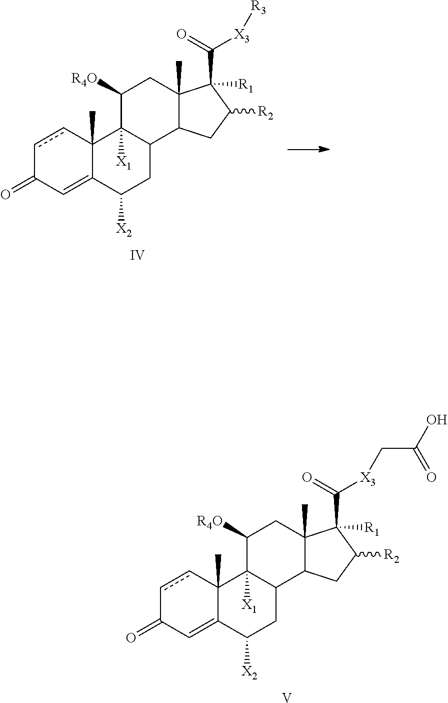
wherein: R₁, R₂, R₃, R₄, X₁, X₂ and X₃ are as defined with reference to formulae (I), (III) and (IV); and/or
(c) reacting a compound of formula (V) with a suitable fluorodecarboxylating agent to yield a compound of formula (VI), 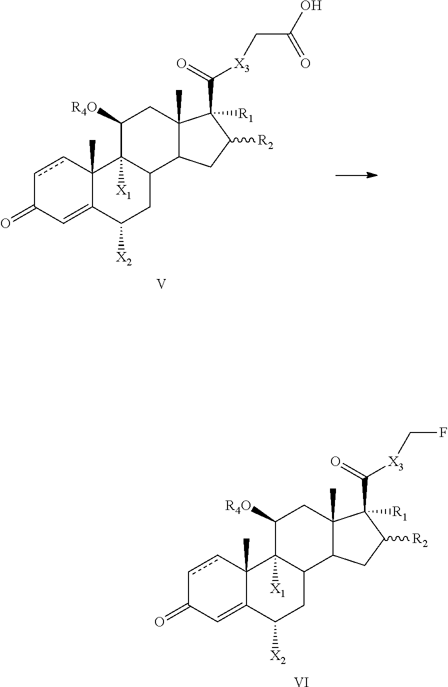
wherein: R₁, R₂, R₄, X₁, X₂ and X₃ are as defined with reference to formulae (I), (III) and (IV); and/or
(d) hydrolysing the C-11 protecting group of a compound of formula (VI) as defined in step (d) to yield a compound of formula (I).

2. A method according to claim 1, wherein:
R₁ is selected from a group consisting of hydroxyl, propionate and furoate; and/or
R₂ is selected from a group consisting of H and methyl; and/or
R₃ is (tert-butylcarboxy)methyl; and/or
R₄ is trifluoroacetate or trichloroacetate; and/or
X₁═X₂═F; and/or
X₃ is S.

3. A method according to claim 1, wherein the fluorodecarboxylating agent is selected from XeF₂ and BrF₃.

4. A method according to claim 1, wherein the organic biologically active of formula (I) is chosen from a group consisting of Fluticasone, Fluticasone Propionate and Fluticasone Furoate.

5. A compound of formula (III), 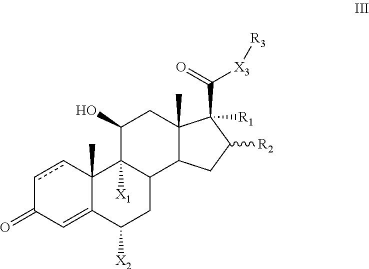
wherein R₁, R₂, R₃, X₁, X₂ and X₃ are as defined in claim 1.

6. A compound of formula (IV), 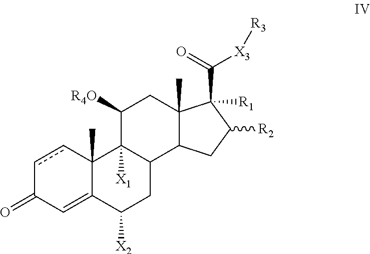
wherein R₁, R₂, R₃, R₄, X₁, X₂ and X₃ are as defined in claim 1.

7. A compound of formula (V), 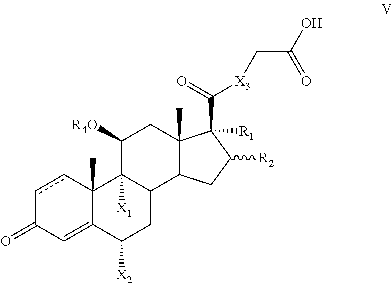
wherein R₁, R₂, R₄, X₁, X₂ and X₃ are as defined in claim 1.

8. A compound of formula (VI), 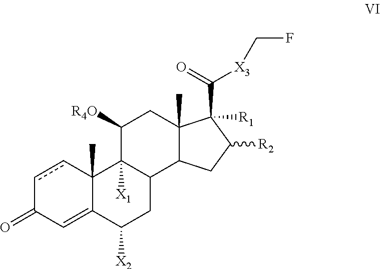
wherein,
R₁ is selected from a group consisting of hydroxyl, propionate and furoate;
R₂ is selected from a group consisting of H and methyl; R₃ is (tert-butylcarboxy)methyl;
R₄ is trifluoroacetate or trichloroacetate; X₁═X₂═F; and
X₃ is S.

9. A method of making an organic biologically active compound containing a “—CH₂F” moiety of claim 1, wherein a compound of formula (III) is used.

10. The method according to claim 9, wherein the organic biologically active compound is a compound of formula (I) as defined in claim 1.

11. The method according to claim 10, wherein the compound of formula (I) is Fluticasone, Fluticasone Propionate or Fluticasone Furoate.

12. A method according to claim 2, wherein the fluorodecarboxylating agent is selected from XeF₂ and BrF₃.

13. A method according to claim 2, wherein the organic biologically active of formula (I) is chosen from a group consisting of Fluticasone, Fluticasone Propionate and Fluticasone Furoate.

14. A method according to claim 3, wherein the organic biologically active of formula (I) is chosen from a group consisting of Fluticasone, Fluticasone Propionate and Fluticasone Furoate.

15. A method of making an organic biologically active compound containing a “—CH₂F” moiety of claim 1, wherein a compound of formula (IV) is used.

16. The method according to claim 15, wherein the organic biologically active compound is a compound of formula (I) as defined in claim 1.

17. The method according to claim 16, wherein the compound of formula (I) is Fluticasone, Fluticasone Propionate or Fluticasone Furoate.

18. A method of making an organic biologically active compound containing a “—CH₂F” moiety of claim 1, wherein a compound of formula (V) is used.

19. The method according to claim 18, wherein the organic biologically active compound is a compound of formula (I) as defined in claim 1.

20. The method according to claim 19, wherein the compound of formula (I) is Fluticasone, Fluticasone Propionate or Fluticasone Furoate.

21. A method of making an organic biologically active compound containing a “—CH₂F” moiety of claim 1, wherein a compound of formula (VI) is used.

22. The method according to claim 21, wherein the organic biologically active compound is a compound of formula (I) as defined in claim 1.

23. The method according to claim 22, wherein the compound of formula (I) is Fluticasone, Fluticasone Propionate or Fluticasone Furoate.
